(19) United States
(12) Patent Application Publication
(10) Pub. No.: US 2011/0294142 A1
(43) Pub. Date: Dec. 1, 2011

(54) Multiply Fluorescently Labeled Calcium Phosphate-Protein Surfaces

(76) Inventors: Joydeep Lahiri, Painted Post, NY (US); Hongwei Hanna Rao, Horseheads, NY (US); Jian Tan, Ithaca, NY (US)

(21) Appl. No.: 12/846,209

(22) Filed: Jul. 29, 2010

Related U.S. Application Data
(60) Provisional application No. 61/349,051, filed on May 27, 2010.

(51) Int. Cl. G01N 33/574; B05D 1/36; G01N 33/50

(52) U.S. Cl. 435/7.23; 435/7.2; 435/7.21; 427/414

(57) ABSTRACT

Described herein are multiply-fluorescently-labeled calcium phosphate protein substrates composed of a base coated with a fluorophore-labeled calcium phosphate coating and further having a fluorescently-labeled protein. The multiply-fluorescently-labeled substrate may be a fluorescently labeled calcium phosphate surface having a fluorescently-labeled collagen. The substrates are useful in culturing and studying the activity of a variety of cells, including bone cells. The substrates described herein can be used for both solution- and image-based analysis of cultured cells. New methods for producing and using such coated substrates are also disclosed.

### CROSS-REFERENCE TO RELATED APPLICATIONS

This application claims the benefit of priority under 35 U.S.C. §119(e) of U.S. Provisional Application Ser. No. 61/349,051 filed on May 27, 2010.

### BACKGROUND

Culturing of adherent animal cells is generally carried out by seeding a substrate with cells in the presence of a biological medium. The cell culture substrate and medium are important in providing an environment in which cells adhere and function in a manner that is predictive of in vivo cell function. Cell culture can provide a research tool for studying diseases and possible drugs for treating or preventing these diseases. When cells are cultured in conditions that allow the cells to be predictive of in vivo cell function, cell culture is more valuable.

When cells are cultured on the surface of a substrate, the cells can be imaged by an optical microscope. Image based analysis, however, can be tedious and may not be possible in real time. For example, when bone cells are cultured, it is often desirable to examine pits formed on the surface of a resorbable cell culture substrate to determine characteristics of bone cells in culture. To examine these pits, cell culture is stopped and cell culture surfaces are treated to reveal pits formed on the surface of the substrate by the cells in culture. Additional methods can be used in cell culture to analyze characteristics of cells in culture. It may be desirable to carry out multiple methods to analyze the characteristics of cells in culture at the same time, in the same cell culture. Thus, in addition to surface imaging, it would be desirable to have a substrate that permits additional detection techniques when cells are cultured on a substrate. Described herein are substrates and methods for producing cell culture surfaces that address these needs.

### SUMMARY

In accordance with the purposes of the disclosed compositions, articles, and methods, as embodied and broadly described herein are substrates composed of (1) a base coated with a fluorescently-labeled or fluorophore-labeled calcium phosphate coating and (2) a protein provided in or on the base having its own fluorescent label, forming a multiply-fluorescently labeled cell culture surface. In embodiments of the present invention, fluorochrome-labeled calcium phosphate surfaces upon which fluorochrome-labeled proteins are adsorbed. In addition to the fluorescently-labeled or fluorophore-labeled calcium phosphate coating, additional labeled components may be incorporated in or on the substrate. For example, additional biologically relevant proteins, which may be fluorescently labeled separately from the substrate, may be incorporated in or on the substrate in order to provide multiple labels in the cell culture environment, to allow for multiple analyses from one cell culture. The substrates are useful in culturing and studying the activity of a variety of cells. The substrates described herein can be used for both solution- and image-based analysis of cultured cells. New methods for producing and using such multiply-fluorescently labeled substrates are also disclosed.

In an aspect (1) a substrate comprising a fluorophore-labeled calcium phosphate coating on the surface of a base is provided. In an aspect (2), the fluorophore is a calcium chelating fluorophore. However, the fluorophore is not limited to calcium chelating fluorophores, it can be any material that fluoresces and stay in the coating or base. In an aspect (3), the substrate of aspect 1, wherein the fluorophore is selected from the group consisting of calcein, xylenol orange, calcein blue, alizarin complexone, doxycycline, oxytetracycline, rolitetracycline, hematophrophyrin and BAPTA (BAPTA: 1,2-Bis (2-aminophenoxy) ethane-N,N, N′, N′-tetraacetic acid) is provided. In an aspect (4), the substrate of aspect 1, wherein the fluorophore is calcein, xylenol orange or calcein blue is provided. In an aspect, (5), the substrate of aspect 1, wherein the calcium phosphate coating is labeled with a plurality of fluorophores is provided. In an aspect (6), the substrate of aspect 5 wherein the plurality of fluorophores are selected from the group consisting of calcein, xylenol orange, calcein blue, alizarin complexone, doxycycline, oxytetracycline, rolitetracycline, hematophrophyrin and BAPTA (BAPTA: 1,2-Bis (2-aminophenoxy) ethane-N,N, N′, N′-tetraacetic acid) is provided. In an aspect (7), the substrate of aspect 5 wherein the plurality of fluorophores comprise calcein and one or more of xylenol orange, calcein blue, alizarin complexone, doxycycline, oxytetracycline, rolitetracycline, hematophrophyrin and BAPTA (BAPTA: 1,2-Bis (2-aminophenoxy) ethane-N,N, N′, N′-tetraacetic acid) is provided. In an aspect (8), the substrate of aspect 5 wherein the plurality of fluorophores comprise xylenol orange and one or more of calcein, calcein blue, alizarin complexone, doxycycline, oxytetracycline, rolitetracycline, hematophrophyrin and BAPTA (BAPTA: 1,2-Bis (2-aminophenoxy) ethane-N,N, N′, N′-tetraacetic acid) is provided. In an aspect (9), the substrate of aspects 1-8 wherein the calcium phosphate coating comprises hydroxyapatite or substituted hydroxyapatite is provided. In an aspect (10), the substrate of aspects 1-8, wherein the base comprises polystyrene, polypropylene, polycarbonate, polyester, or any combination thereof is provided. In an aspect (11), the substrate of aspects 1-8, wherein the base comprises an inorganic material is provided. In an aspect (12), the substrate of aspect 11, wherein the inorganic material comprises glass, quartz, ceramic, silica, a metal oxide, or any combination thereof is provided. In an aspect (13), the substrate of aspects 1-8, wherein the base comprises a microwell, dish, or flask is provided. In an aspect (14), the substrate of aspect 1, wherein fluorophore-labeled calcium phosphate coating on the surface of a base is prepared by a method comprising: (a) introducing the base into a solution comprising a plurality of precursor components for producing the calcium phosphate coating and a fluorphore; (b) inverting the base relative to the solution; and (c) incubating the inverted base to produce the fluorophore-labeled calcium phosphate coating on the surface of the base is provided. In an aspect (15), the substrate of aspect 14, further comprising, after step (c), exposing the fluorphore-labeled calcium phosphate coating on the base to gamma irradiation is provided. In an aspect (16), a method for producing a fluorophore-labeled calcium phosphate coating on the surface of a base, the method comprising providing a fluorophore to a calcium phosphate coating on the surface of the base is provided. In an aspect (17) A method for evaluating the activity of a cell or cell precursor, the method comprising: (a) culturing cells or cell precursors in a culture medium on the substrate of claim any one of aspects 1 through 13; (b) exposing the cultured cells on the substrate to a wavelength of light which corresponds to the fluorescence of the fluorophore, (c) characterizing the fluorescence of the fluorophore is provided. In an aspect (18), the method of aspect 17, wherein cell comprises stem cells, committed stem cells, differentiated cells, tumor cells, myoblasts, neuroblasts, fibroblasts, glioblasts, germ cells, hepatocytes, chondrocytes, keratinocytes, smooth muscle cells, cardiac muscle cells, connective tissue cells, glial cells, epithelial cells, endothelial cells, hormone-secreting cells, cells of the immune system, or neurons is provided. In an aspect (19), the method of aspect 17, wherein the cell is a bone cell, and the bone cell is an osteoclast, an osteocyte, or an osteoblast is provided. In an aspect (20), the method of aspect 17, wherein the cell precursor is a bone cell precursor, and the bone cell precursor is monocyte or a macrophage is provided. In an aspect (21) the method of aspect 17, wherein characterizing comprises quantifying resorption pits produced by a bone cell or bone cell precursor is provided. In an aspect (22), the method of aspect 17, wherein the base comprises a polymer comprising polystyrene, polypropylene, polycarbonate, polyester, or any combination thereof is provided. In an aspect (22), the method of aspect 17, wherein the base comprises a glass slide, wherein a gasket is adhered to the slide to provide at least one temporary well, and adding solution to the well or wells is provided.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying figures, which are incorporated in and constitute a part of this specification, illustrate several aspects described below.

FIG. 1 is a schematic cross-section of an embodiment of the “pre-substrate.”

FIG. 2 is a schematic cross-section of an embodiment of a multiply fluorescently-labeled calcium phosphate and protein substrate.

FIGS. 3 A, B and C are scanning electron micrographs (SEMS) of calcein labeled calcium phosphate surfaces labeled with 20 μm, 30 μm and 80 μm calcein mixed with 5× simulated body fluid (SBF) respectively.

FIGS. 4 A, B and C are scanning electron microscope (SEM) photographs of Xylene Orange (XO) labeled calcium phosphate surfaces labeled with 10 μm, 50 μm and 100 μm XO mixed with 5×SBF respectively.

FIG. 5 is a graph illustrating the measurement of EU-labeled collagen in culture medium (measured by RFU) during culture of human osteoclast precursor cells on an embodiment of a cell culture surface having Europium-labeled collagen adsorbed on calcein-labeled calcium phosphate surfaces, for up to 7 days.

FIGS. 6A, B and C are a photographs of europium-labeled collagen adsorbed on calcein-labeled calcium phosphate cell culture surfaces on day 1 (FIG. 6A), day 6 (FIG. 6B) and day 7 (FIG. 6C) of cell culture. After 6 and 7 days of culture, pit formation is visible. While not shown in black and white, the photographs show green surfaces.

### DETAILED DESCRIPTION

In embodiments of the present invention, fluorochrome-labeled calcium phosphate surfaces which include fluorochrome-labeled proteins are described, along with methods of making the fluorochrome-labeled calcium phosphate surfaces having fluorochrome-labeled proteins. In embodiments, these fluorochrome-labeled calcium phosphate surfaces having fluorochrome-labeled proteins are suitable for culturing cells, and for performing both solution-based and image-based fluorescent assays to quantitatively analyze activities such as bone resorption activities. In embodiments, multiple solution-based and/or image-based fluorescent assays may be performed on embodiments of the cell culture surface in the same cell culture. In embodiments, these surface provide unique features for quick, direct and specific bone resorption assays in vitro. In embodiments, the fluorescence of the surface is compatible with current existing fluorescent imaging systems.

The materials, compounds, compositions, articles, devices, and methods described herein may be understood more readily by reference to the following detailed description of specific aspects of the disclosed subject matter and the Examples included therein and to the Figures.

Before the present materials, compounds, compositions, articles, devices, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, and reference to “a precursor” includes mixtures of two or more such precursors.

“Optional” or “optionally” means that the subsequently described event or circumstance can or cannot occur, and that the description includes instances where the event or circumstance occurs and instances where it does not.

“Fluorophore” means a compound that fluoresces when exposed to an appropriate wavelength of light. More specifically, a fluorophore is a component of a molecule which absorbs energy of a specific wavelength and re-emits energy at a different wavelength. A fluorophore is a fluorescent label. A fluorophore-labeled composition is a fluorescently-labeled composition, and these terms may be used interchangeably.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers or prepared by methods known to those skilled in the art.

Reference will now be made in detail to specific aspects of the disclosed materials, compounds, compositions, articles, and methods, examples of which are illustrated in the accompanying Examples and Figures.

Approximately 70% of bone is made up of the inorganic mineral calcium phosphate. Calcium phosphate is found in many forms. In bone, the major component is hydroxyapatite (HA). Approximately 30% of bone is composed of organic components, mainly Type I collagen. Type I collagen constitutes approximately 95% of the bone organic matrix; the remaining 5% is composed of proteoglycans and other non-collagenous proteins. Bone is a dynamic organ. Bone is constantly remodeled by bone-forming osteoblasts and bone-resorbing osteoclasts. Bone resorption is the process by which osteoclasts break down bone and release inorganic minerals, mainly calcium ions and organic components, which is mostly degraded collagen fragments, to the extracellular space. Bone resorption also makes resorption pits on the bone surface. Osteoclasts are the cells responsible for resorbing both the organic and inorganic components of bone.

There are two major categories of methods to assess osteoclast bone resorption activities in vitro. One category of methods is solution-based. It includes measuring the amount of calcium ion or degraded collagen fragments transported extracellularly in the solution of cell culture medium to quantify the functional activities of osteoclasts. Another category of methods are image-based assays involving the image analysis of resorption pits on natural or synthetic bone surface to measure the functional activities of osteoclasts.

An imbalance in the function of these cell types causes bone related diseases such as osteoporosis. Osteoporosis is a major public health problem, and is becoming increasingly prevalent with the aging of the world population. Most bone diseases are due to increased bone resorption, thus the osteoclast is the main target for the pharmaceutical industry to find therapeutic solutions for bone diseases. Advances in bone cell biology research has enabled drug discovery for treating and preventing osteoporosis and the implication of bone related diseases.

There are two major categories of methods to assess osteoclast bone resorption activities in vitro. One category of methods is solution-based. It includes measuring the amount of calcium ion or degraded collagen fragments transported extracellularly in the solution of cell culture medium to quantify the functional activities of osteoclasts.

For example, in U.S. Pat. No. 5,834,221, a method for determining collagen degradation in vivo is described, comprising quantification of the concentration of a peptide derived from the carboxy-terminal telopeptide domain of the al (I) chain of type I collagen in a body fluid including urine, serum, or synovial fluid.

A second category of methods are image-based assays. For example, when osteoclasts are grown in culture on bone material, the cells resorb the bone material. In culture, these areas of resorption are called resorption pits. Bone resorption can be measured by measuring the size and characterizing resorption pits that form when cells (osteoclasts) grown in culture resorb bone material. Osteoclast functional activities can be assessed in cell culture by measuring and characterizing bone resorption pits formed on natural or synthetic bone surfaces.

For example, OsteoAssay™ Human Bone Plates (available from Lonza (Walkersville, Pa.) use a thin layer of human bone particles adherent to cell culture plates. The functional octeoclasts cultured on OsteoAssay™ Human Bone Plates degrade the bone particles and release degraded collagen fragments in cell culture supernatant. The bone resorption activities of osteoclasts can be measured by determining the amount of released calcium or degraded collagen fragments in the cell culture supernatant after an appropriate period of cell culture. However, OsteoAssay™ Human Bone Plates cannot provide images of bone resorption pits due to the small size of ground human bone particles.

Osteologic™ slides or discs from BD Biosciences (Franklin Lakes, N.J.), containing a layer of sintered calcium phosphate, are designed for image-based bone resorption assays. OAAS™ surfaces are available form Oscotec (Choongnam, Republic of Korea). Similarly, animal bone slices and dentine have been used for image-based bone resorption assays.

Corning Incorporated has developed an Osteo Assay™ Surface, which is a thin layer of calcium phosphate crystals coating on corona treated or tissue culture treated (TCT) polystyrene substrate. Corning Osteo Assay™ Surface can provide image-based bone resorption assays.

Another newly developed surface, described in U.S. patent application Ser. No. 12/625,952, also incorporated herein in its entirety discloses, among other embodiments, europium labeled collagen on unlabeled calcium phosphate crystals on top of a TCT polystyrene substrate, can provide both solution-based and image-based bone resorption assays. However, the image-based resorption assay from that surface cannot be used for real-time measurements. Using that surface, one must start with multiple cell cultures and terminate them one by one at certain time intervals to treat or stain the surface in order to acquire images of bone resorption pits. Therefore, bone resorption data collected using this surface must be collected from many different cell cultures.

To study the kinetics of bone resorption, researchers have had to start with multiple cell cultures, and terminate them one-by-one at a certain time intervals over a period of time; therefore, the results are from many different cell cultural wells. In addition, at each time interval, cell manipulation or treatment is often required for the assay.

Co-pending U.S. patent application Ser. No. 12/846,043 discloses and claims a fluorescently-labeled calcium phosphate surface. The surfaces disclosed in that application can be used to visualize the formation of resorption pits during cell culture, as shown by a change in the color of the fluorescently-labeled calcium phosphate surface, when a cell resorbs the fluorescently labeled calcium phosphate cell culture surface. That is, in embodiments, the surfaces may be used for image-based resorption assays without terminating cell culture. In embodiments, the multiply-fluorescently labeled calcium phosphate-protein surfaces provided herein may be used for both solution-based and image-based fluorescent assays to quantitatively analyze bone resorption activities of osteoclasts. That is, in embodiments, the activity of cells in culture to break down fluorophore-labeled collagen can be measured by measuring the presence of collagen-fluorophore in solution during cell culture. At the same time, the activity of cells in culture to break down calcium phosphate can be measured by measuring the presence and characteristics of resorption pits, which are measureable by analyzing changes in fluorophore-labeled calcium phosphate surfaces during cell culture. In embodiments, methods for making calcium-binding fluorochrome labeled calcium phosphate and europium labeled collagen (Eu-collagen) on corona treated or polystyrene surfaces are disclosed.

Calcium-binding fluorescent dyes have been used to assess bone formation and remodeling in animal model (Pautkea C., Vogtb S., Tischerb T., Wexelc G., Deppea H., Milzd S., Schiekere M., Kolka A. Polychrome labeling of bone with seven different fluorochromes: Enhancing fluorochrome discrimination by spectral image analysis. Bone 37, (2005), pp 441-445). Mineralized bone nodule formation can also be examined in living osteoblastic cultures using fluorescent dyes (Wang Y H., Liu Y., Maye P., Rowe D W. Examination of Mineralized Nodule Formation in Living Osteoblastic Cultures Using Fluorescent Dyes. Biotechnol. Prog. 22, (2006), pp 1697-1701).

OsteoImage™ from Lonza Walkersville Inc is based on the specific binding of OsteoImage staining reagent to the hydroxyapatite of the bone nodule deposited by the cells Lonza Walkersville Inc. (WWW.lonza.com—OsteoImage™ Mineralization Assay Instructions for Use).

Table 1 shows a group of fluorochromes such as calcein, xylenol orange (XO), calcein blue which can chelate calcium ion and fluoresce at different excitation and emission wave lengths.

TABLE 1

| | Max. Excitation | Max. Emission | |
|---|---|---|---|
| Flurochrome | (nm) | (nm) | Color |
| Calcein | 494 | 517 | Green |
| Xylenol orange | 440 | 610 | Red |
| Calcein blue | 373 | 420-440 | Blue |
| Alzarin | 530-580 | 624-645 | Red |
| complexone | | | |
| Doxycycline | 390-425 | 520-560 | Yellow |
| Oxytetracycline | 390-425 | 520-560 | Yellow |
| Rolitetracycline | 390-425 | 520-560 | Yellow |
| Hematophrophyrin | 530-560 | 580 | Red |
| BAPTA* | 200-325 | 410-550 | Purple |

*BAPTA: 1,2-Bis(2-aminophenoxy)ethane-N,N,N′,N′-tetraacetic acid

In embodiments of the present invention a fluorescent synthetic bone surface incorporating calcium binding fluorescent dyes for osteoclast bone resorption assays are described. In embodiments, the surfaces not only provide a substrate for traditional image based bone resorption assays, they are also suitable for image based real time assays to measure bone resorption.

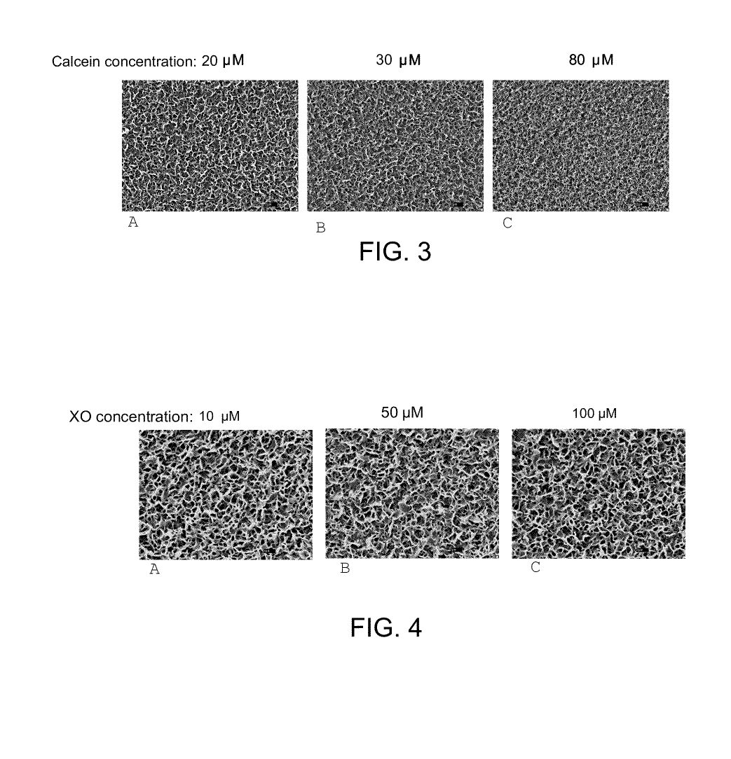

The chemical formula for calcein, which fluoresces in green at 494/517 nm is shown in Formula 1. EM photographs of fluorescently labeled calcium phosphate surfaces, labeled with calcein are shown in FIG. 3. The chemical formula for xylenol orange (XO) which fluoresces red at 440/570 nm is shown in Formula 2. A scanning electron micrograph of a fluorescently labeled calcium phosphate surface, labeled with XO is shown in FIG. 4. The chemical formula for calcein blue (CB) which fluoresces blue at 375/517 nm is shown in Formula 3. A fluorescently labeled calcium phosphate surface, labeled with CB appears blue (not shown).

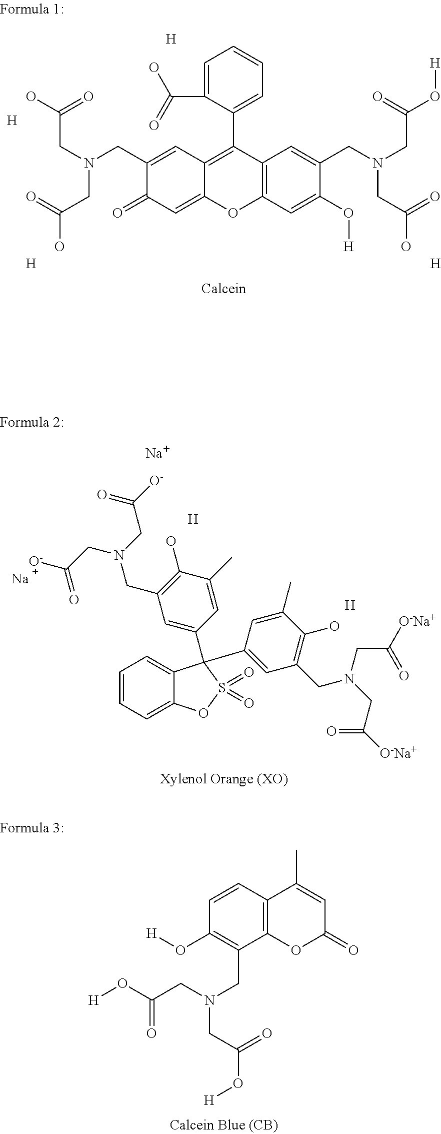

Described herein are substrates comprising a fluorophore-labeled calcium phosphate coating on the surface of a base. In embodiments, a fluorescently labeled (or fluorophore-labeled) calcium phosphate coating, which may be, in embodiments, a calcium phosphate coating is described. In embodiments, the fluorescent calcium phosphate cell culture surface is suitable for osteoclast bone resorption assays. In embodiments, methods for making fluorescently labeled calcium phosphate coatings are described. In embodiments, multiple fluorophores may be used to fluorescently label the fluorescently labeled calcium phosphate surface forming a “pre-surface.” For example, in embodiments, the pre-surface may be labeled with one fluorophore, two fluorophores, three fluorophores or more fluorophores. In additional embodiments, the pre-surface have a fluorophore labeled protein absorbed on a surface of the pre-surface. In additional embodiments, methods for making fluorescently labeled calcium phosphate coatings on tissue culture treated polystyrene plates for osteoclast bone resorption assay are described. In embodiments, fluorescently labeled calcium phosphage coatings provide cell culture surfaces suitable for real time course image based bone resorption assay. In embodiments, the pre-surface with the absorbed fluorophore-labeled calcium phosphate fluorophore labeled protein is not cytotoxic, and provides a surface to which cells can adhere. In embodiments, the calcium phosphate coating is a hydroxyapatite coating.

Each component of the substrate as well as methods for making and using the substrates are described in detail below.

The term “base” as used herein is any article having a surface where calcium phosphate can be coated. In one aspect, the substrate can possess one or more wells or depressions that can receive and hold a solution that can produce a calcium phosphate coating. The base can assume many shapes and sizes depending upon the desired end-use of the multi-purpose substrate. For example, the base can be a microwell plate having a plurality of wells with varying diameters and heights.

The base can be prepared from a variety of different materials. In one aspect, the base comprises a polymer. Examples of such polymers include, but are not limited to homopolymers and copolymers of a polyester, a polyvinylchloride, a polyvinylidene fluoride, a polytetrafluoroethylene, a polycarbonate, a polyamide, a poly(meth)acrylate, a polystyrene, a polyethylene, polypropylene, or an ethylene/vinyl acetate copolymer. Blends of polymers are also known and may also be considered for this application. These blends may include, but are not be limited to commercially available materials such as polycarbonate/ABS, PVC/ABS, polyphenyleneoxide and high impact polystyrene, but also may include novel blends of the homopolymers and copolymers listed above. These polymers can be formed into cell culture vessels including wells, multi-well plates, flasks, and the like. In addition, the cell culture container may be a virtual well formed from a bottom base, such as a glass slide, or a sheet of polymer material, with a structure placed upon the bottom base in a water-impermeable manner, to form sidewalls of a cell culture well.

In one aspect, the polymeric base can be modified prior to applying the calcium phosphate coating. The polymeric base can be modified to change the charge of the base, to include active chemical moieties, or to increase the amount of surface oxygen. For example, the surface of the base can be exposed to energy such as corona discharge, plasma treatment (e.g., ammonia, nitrogen, oxygen, nitrous oxide, carbon dioxide, air, or other gases that can be activated or ionized), heat, ultraviolet radiation, gamma radiation, UV ozone, or microwave energy. The increase in surface oxygen increases the hydrophilic nature of the base, which can be desirable in certain aspects. The treatment of the base surface can also modify the overall surface charge on the base, which can facilitate coating the surface with calcium phosphate. In one aspect, the base comprises polystyrene that has been treated to increase the amount of surface oxygen.

In another aspect, the base comprises an inorganic material. Examples of inorganic materials include metals and semiconductor materials that can be surface oxidized, glass, and ceramic materials. Examples of metals that can be used as base materials are oxides of aluminum, chromium, titanium and steel. Semiconductor materials used for the base material can include silicon and germanium. Glass and ceramic materials used for the base material can include quartz, glass, porcelain, alkaline earth aluminoborosilicate glass, soda lime silicate glass and other mixed oxides. Further examples of inorganic substrate materials include zinc compounds, mica, silica and inorganic single crystal materials. It is contemplated that the base can include be a layered system, any of the polymeric or inorganic materials described above can be coated on each other. For example, the base can be a polymeric surface coated with silica. Or, for example, the base can be contacted with a solution comprising a plurality of precursor components for producing the calcium phosphate coating. The method of contacting the base with the solution varies with the selection of the base. For example, when the base is a glass slide, the slide can be adhered to a gasket (e.g., flexiPerm reusable cell culture chamber manufactured by Greiner Bio One, Germany), and the solution is added to the wells. In another aspect, when the base is a microwell plate, each well is filled with a specific amount of solution. In this aspect, it is contemplated that each well is filled with the same or different solution (i.e., different precursor components and/or different amounts of precursor components). The amount of solution added to each well can vary, and will depend upon the size of the well (diameter and height), the material of the base, and the concentration of the precursor components. In one aspect, each well is partially filled with the solution. Another technique for contacting the base with the solution is spray coating.

The precursor component is any component that can result in the formation of a calcium phosphate coating on the surface of the base. Although the precursor component is generally a salt or a mixture of salts, the precursor component can be an acid or base as well. In one aspect, the precursor component comprises an alkali metal halide, an alkali metal sulfate, an alkali metal carbonate, an alkali metal phosphate, an alkaline earth metal halide, an alkaline earth metal sulfate, an alkaline earth metal carbonate, an alkaline earth metal phosphate, or any combination thereof. It is intended that carbonate also includes bicarbonate phosphate also includes hydrogen and dihydrogen phosphate, and sulfate also includes hydrogen sulfate.

In another aspect, the precursor component comprises any combination of calcium chloride, magnesium chloride, sodium bicarbonate, potassium hydrogen phosphate, sodium phosphate, and sodium chloride. The ions of these components are generally present in blood plasma. Thus, solutions comprising these components are generally referred to as simulated body fluids or SBF. The production of simulated body fluids or derivatives thereof is known in the art. The SBF solutions can be modified. For example, in certain aspects, the solution does not require potassium or sulfur. An example of components of 5×SBF solutions are presented in Table 2.

TABLE 2

| Component | Chemical | Grams/Liter |
|---|---|---|
| 1 | NaCl | 39.98 |
| 2 | CaCl₂H₂O | 1.84 |
| 3 | MgCl₆H₂O | 1.53 |
| 4 | NaHPO₄H₂O | 1.09 |

The concentration of precursor components present in the solution can vary. In certain aspects, the concentration is the maximum amount of precursor components that are soluble in water alone or in combination with minor amounts of other solvents (e.g., an alcohol) or pH modifiers (e.g., acids or bases). In one aspect, the solution comprises SBF. In another aspect, the solution comprises 5× or 10×SBF. The initial pH of the solution can also vary with the concentration of precursor components, the material of the base, and the surface charge (if any) on the base surface. In one aspect, the solution has an initial pH of from 3 to 8, 3 to 7, 3 to 6, 4 to 8, 5 to 8, 5 to 7, or 5 to 6. By varying the pH of the solution, it is possible to control the overall morphology of the calcium phosphate coating formed on the base.

In embodiments, a fluorophore or a fluorescent dye may be added to the precursor components. For example, a fluorophore may be added to the SBF precursor components. The fluorophore may be added at different concentrations. For example, in embodiments, a single fluorophore may be added at a concentration of from 5 to 300 μM, at a concentration of 5 to 200 μM, 5 to 100 μM, 5 to 50 μM, 5 to 25 μM, 10 to 300 μM, 10 to 200 μM, 10 to 50 μM, 10 to 25 μM, 50 to 300 μM, 50 to 200 μM, 50 to 100 μM, or any suitable range. In additional embodiments, multiple fluorophores may be added to the precursor components.

As discussed above, in certain aspects the base can be treated with a number of surface techniques to change the surface charge of the base, which in turn can influence surface wettability. For example, when the surface of the base is treated to increase the amount of oxygenated groups (e.g., hydroxyl, carboxyl), the treated base may have a greater affinity for the solution. Another consideration is the amount of solution used. In general, the volume of solution is sufficient to produce a suitable calcium phosphate coating. The amount of solution used depends on the concentration of the solution and the desired thickness of the coating.

After the base has been contacted with the solution, the base is incubated to produce the calcium phosphate coating on the surface of the base. The calcium phosphate coating on the surface of the base is the “pre-substrate.” The temperature and duration of incubation can vary depending upon the desired morphology of the calcium phosphate coating on the base. For example, it may be desirable to have a longer incubation time at a lower temperature to produce smaller calcium phosphate crystals on the surface of the base. In one aspect, the incubation step is performed at a temperature up to 90° C. for up to 72 hours. In another aspect, the incubation step is performed at a temperature from room temperature to 90° C., 30° C. to 80° C., or 40° C. to 60° C. from 1 to 72 hours, 2 to 36 hours, 2 to 24 hours, or 2 to 18 hours.

Depending upon the selection of the precursor components, gases can be produced during incubation and crystallization. For example, if the solution comprising the precursor components is acidic and bicarbonate is added to the solution, CO₂ gas is produced. Not wishing to be bound by theory, the removal of the gas can influence the pH of the solution, which in turn can influence the rate and amount of crystal formation. Depending upon the components present in the solution, crystal formation may be sensitive to changes in pH. For example, when bicarbonate is added to an acidic solution, CO₂ is generated. If CO₂ is removed from the system, the equilibrium is shifted to the right and more acid in solution is removed (i.e., reacts with bicarbonate). This results in an increase in pH. If CO₂ is not removed, an equilibrium is reached, and no further change in acid concentration and pH occurs (i.e., bicarbonate does not react any further with the acid). Thus, where crystal growth on the base is sensitive to the pH of the solution, removal of any gases generated during incubation can be performed to promote crystal formation.

In other aspects, when the base comprises a series of bases (e.g., a stack of microplates or Petri dishes), during incubation a slight vacuum can be applied to remove gas from the stacked system. In the alternative, the stacked system can be arranged such that the each plate or dish is loosely stacked so that any gases generated during incubation can escape. In embodiments, the base may be inverted during the incubation step.

After the incubation step, the base has a calcium phosphate coating. Subsequent steps can be performed on the coated base including washing with water and drying by applying a stream of air or heating the substrate.

The thickness of the calcium phosphate coating on the base can vary depending upon the base to be coated as well as the nature and concentration of precursor components selected. For example, the thickness of the coating ranges from 200 nm to 800 nm, 200 nm to 700 nm, 200 nm to 600 nm, 200 nm to 500 nm, 200 nm to 400 nm, 300 nm to 800 nm, 400 nm to 800 nm, 500 nm to 800 nm, or 600 nm to 800 nm. If thicker coatings are desired, the contacting and incubation steps described above can be performed multiple times sequentially to produce thicker coatings. In certain aspects, thinner coatings are desirable (e.g., less than one micron) in order to better visualize the cells on the substrate and improve sensitivity to cell resorption.

The base can be coated with calcium phosphate in a variety of patterns and designs. For example, a removable adhesive tape or mask can be placed on the surface of the base to produce a pattern or design of exposed substrate that ultimately will be coated with calcium phosphate. The tape or mask is then removed after incubation and crystal formation. Alternatively, if the base is to be treated in order to increase surface oxygen, prior to surface treatment, a removable adhesive tape or mask can be placed on the surface of the base. Here, the calcium phosphate coating forms only on the portions or section of the base that have been surface treated, or if crystals form on masked areas they are more easily removed during subsequent washing steps.

In one aspect, the calcium phosphate coating comprises hydroxyapatite, which has the formula Ca₅(PO₄)₃OH. In another aspect, the calcium phosphate coating is composed of a substituted hydroxyapatite. A substituted hydroxyapatite is hydroxyapatite with one or more atoms substituted with another atom. The substituted hydroxyapatite is depicted by the formula M₅X₃Y, where M is Ca, Mg, Na; X is PO₄ or CO₃; and Y is OH, F, Cl, or CO₃. Minor impurities in the hydroxyapatite structure may also be present from the following ions: Zn, Sr, Al, Pb, Ba. In another aspect, the calcium phosphate comprises a calcium orthophosphate. Examples of calcium orthophosphates include, but are not limited to, monocalcium phosphate anhydrate, monocalcium phosphate monohydrate, dicalcium phosphate dihydrate, dicalcium phosphate anhydrous, octacalcium phosphate, beta tricalcium phosphate, alpha tricalcium phosphate, super alpha tricalcium phosphate, or tetracalcium phosphate. In certain aspects, the calcium phosphate coating includes crystals possessing carbonate groups (CO₃), which can facilitate adhesion of certain types of cells such as, for example, bone cells, during culturing. In other aspects, the calcium phosphate coating can also include calcium-deficient hydroxyapatite, which can preferentially adsorb proteins useful in cell culturing such as bone matrix proteins or collagen.

The calcium phosphate coatings produced generally have a high surface area and pore volume. The calcium phosphate coating is generally uniform on the surface of the base, which is desirable for cell culturing. Moreover, when the calcium phosphate coating has a uniform thickness, it enables better evaluation of adherent cells.

In one aspect, the substrate is produced by the method comprising: (a) introducing a base into a solution comprising a plurality of precursor components including a fluorophore for producing a fluorophore-labeled calcium phosphate coating on the surface of the base; (b) inverting the base relative to the solution; and (c) incubating the inverted base to produce the fluorophore-labeled calcium phosphate coating on the surface of the base, wherein gas generated during incubation is permitted to escape.

The methods disclosed in International Publication No. WO 2008/103339, which are incorporated by reference in their entirety, can be used in this aspect. In this aspect, once the base has been contacted with the solution, the base is inverted relative to the solution. For example, when the base is a microwell or a slide adhered to a gasket, the base can be inverted 180°.

In additional embodiments, a fluorophore-labeled calcium phosphate surface is made by coating a pre-made calcium phosphate surface with one or more fluorophores. In embodiments, the fluorophore may be introduced to the pre-made HA surface in concentrations of from 0.075-750M.

In additional embodiments, a fluorophore-labeled calcium phosphate may be made by coating a pre-made fluorophore-labeled calcium phosphate surface with a more than one fluorophore to create a multiple-fluorophore-labeled calcium phosphate surface. The term “adsorbed” as used herein with respect to the fluorophore-labeled calcium phosphate surfaces is defined as the non-covalent attachment or bonding of the fluorophore-label to the calcium phosphate coating.

In another aspect, the fluorophore can be calcein, xylenol orange, calcein blue, alizarin complexone doxycycline, oxytetracycline, rolitetracycline, hematophrophyrin, BAPTA (BAPTA: 1,2-Bis (2-aminophenoxy) ethane-N,N,N′,N′-tetraacetic acid). In embodiments, the fluorochromes react with calcium cations in the calcium phosphate surface to form a chelate. In embodiments, the calcium/fluorochrome chelate is very stable. The stability of these fluorescently labeled calcium phosphate surfaces allows these surfaces to be suitable for storage in dry conditions, as well as use in wet conditions as surfaces for cell culture.

After the fluorophore-labeled calcium phosphate coating has been applied to the base, the substrate can be subsequently washed and dried. In certain aspects, the substrate may be exposed to gamma irradiation.

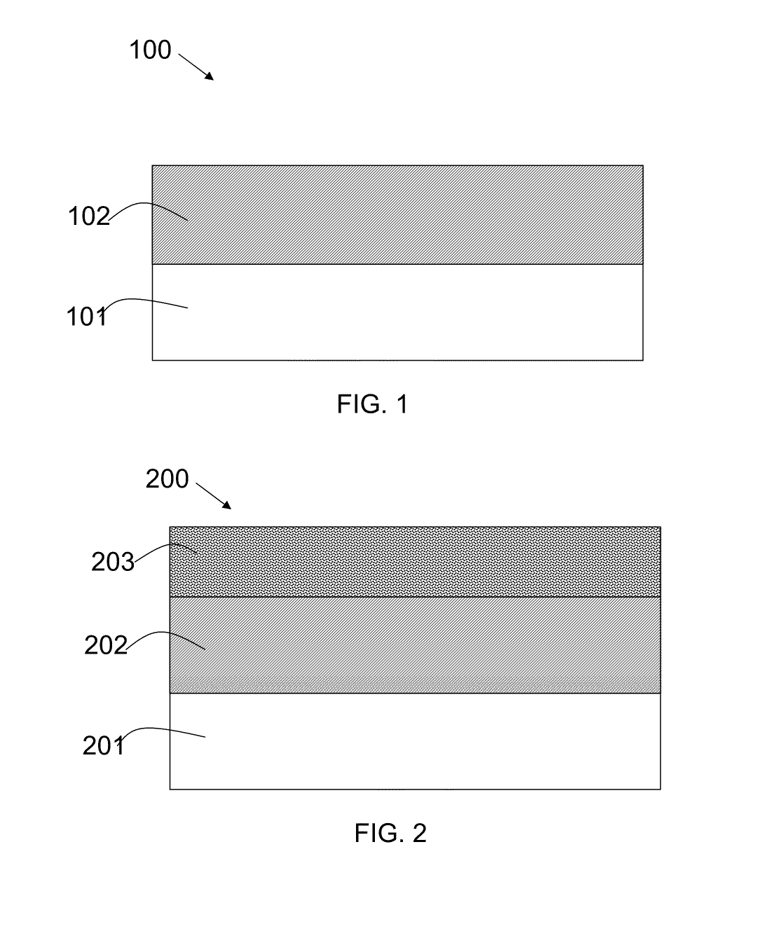

In embodiments, the fluorophore-labeled calcium phosphate coated cell culture coating on a surface of a base is a “pre-substrate.” FIG. 1 is a schematic cross-section of an embodiment of the pre-substrate 100, illustrating the base 101 with a fluorescently-labeled calcium phosphate coating 102.

In embodiments, the cell culture substrates described herein have fluorophore-labeled protein, which may be collagen on the fluorophore-labeled calcium phosphate coated cell culture coating applied to a base. Although the protein may be attached to the pre-substrate by any means, for example by covalent or non-covalent bonds, by hydrophobic/hydrophilic forces, by Vander Waals forces, in embodiments the protein is absorbed to the pre-substrate. The term “substrate” for the purposes of this disclosure, refers to a multiply fluorescently-labeled calcium phosphate protein substrate. That is, a substrate is a multiply fluorescently-labeled calcium phosphate protein substrate, or a base having a fluorescently-labeled calcium phosphate coating and also having a fluorescently labeled protein. FIG. 2 is a schematic cross-section of an embodiment of a multiply fluorescently-labeled calcium phosphate protein substrate. FIG. 2 illustrates the substrate 200 which is a base 201, having a fluorescently-labeled calcium phosphate coating 202, and a fluorescently-labeled protein 203.

In embodiments, the fluorescently-labeled protein may be collagen. In embodiments, the fluorescently-labeled collagen may be “adsorbed” to the fluorescently-labeled calcium phosphate coating. The term “adsorbed” as used herein with respect to the fluorophore-labeled collagen is defined as the non-covalent attachment or bonding of the fluorophore-labeled collagen to the fluorescently labeled calcium phosphate coating. The fluorophore-labeled collagen is neither directly nor indirectly covalently bonded to the calcium phosphate coating. In embodiments, the fluorophore-labeled calcium phosphate coating is not modified in any way so that is can form a covalent bond with the fluorophore-labeled collagen. For example, the fluorophore-labeled collagen is not derivatized with linkers or spacers such as carbodiimides (e.g., 1-ethyl-3-[dimethylaminopropyl]carbodiimide hydrochloride), maleimides, or iodoacetyl groups, which can potentially form covalent bonds with functional groups present on the calcium phosphate coating. In additional embodiments, the fluorophore-labeled collagen may be deriviatized with linkers or spacers such as carbodiimides (e.g., 1-ethyl-3-[dimethylaminopropyl]carbodiimide hydrochloride), maleimides, or iodoacetyl groups, which can potentially form covalent bonds with functional groups present on the fluorescently-labeled calcium phosphate coating.

A wide variety of fluorophores can be used to label a protein. In embodiments, the fluorophore used to fluorescently-label the calcium phosphate coating may be different from the fluorophore used to fluorescently label collagen. In embodiments, the fluorophore used to fluorescently label the calcium phosphate coating is the “calcium phosphate fluorophore” and the fluorophore used to fluorescently label the collagen is the “collagen fluorophore.” The collagen fluorophore can be covalently or non-covalently bound to the collagen. In one aspect, the collagen fluorophore is a lanthanide chelate. Examples of lanthanide chelates useful herein include, but are not limited to, 0-diketone chelates of cerium, praseodymium, neodymium, promethium, samarium, europium, gadolinium, terbium, dysprosium, holmium, erbium, thulium, or ytterbium. Suitable 0-diketones are, for example, 2-naphthoyltrifluoroacetone (2-NTA), 1-naphthoyltrifluoroacetone (1-NTA), p-methoxybenzoyltrifluoroacetone (MO-BTA), p-fluorobenzoyltrifluoroacetone (F-BTA), benzoyltrifluoroacetone (BTA), furoyltrifluoroacetone (FTA), naphthoylfuroylmethane (NFM), dithenoylmethane (DTM), and dibenzoylmethane (DBM). In one aspect, the lanthanide chelate is Eu³⁺-N′-(p-isothiocyanatobenzyl) diethylenetriamine-N¹,N²,N³-tetraacetic acid (Perkin-Elmer).

In another aspect, the collagen fluorophore can be Alexa Fluor 350, Alexa Fluor 488, Alexa Fluor 546, Alexa Fluor 555, Alexa Fluor 594, Alexa Fluor 647, Alexa Fluor 680, fluorescein isothiocyanate (FITC), Rhodamine 110, Rhodamine 123, Rhodamine 6G, Rhodamine Green, Rhodamine Red, and Rhodamine B. In other aspects, the collagen fluorophore can be quantum dots, which are semiconductor nanocrystals with size-dependent optical and electronic properties.

The type of collagen in the fluorophore-labeled collagen is not limited to any particular type of collagen. For example, collagen types I, II, III, IV, V, VI, VII, VIII, VIX, or X, etc. can be used herein. The collagen can be recombinant or naturally occurring collagen. In one aspect, the collagen can be vertebrate collagen. In another aspect, the collagen is mammalian collagen such as, for example, human collagen. The type of collagen that is used can vary depending upon the cultured cell type. For example, when osteoclasts or osteoclast precursors are to be assayed, type I collagen can be used in the fluorophore-labeled collagen. Sources of type I collagen include rat tail collagen, bovine dermis collagen, human placental collagen, and kangaroo tail collagen. Alternatively, when tumor cells are to be assayed, type IV collagen can be used. Sources of type IV collagen include human or other mammalian placental collagen and Engelbreth-Holm-Swarm mouse sarcoma collagen.

The collagen can be purified as needed. It is desirable that the collagen be relatively pure so that detected fluorescence reflects true collagen degradation and not degradation of impurities. In one aspect, the collagen is at least about 90% pure, at least about 95% pure, or close to 100% pure.

The adsorption of the fluorophore-labeled collagen to the pre-substrate does not require special techniques or handling. For example, the pre-substrate can be immersed in a solution containing the fluorophore-labeled collagen. The temperature and duration of the adsorption step can vary depending upon the concentration of the fluorophore-labeled collagen and the desired thickness of the fluorophore-labeled collagen that is to be adsorbed on the pre-substrate. In one aspect, the adsorbing step is performed at 0° C. to 60° C. from 1 to 4 hours. In another aspect, the amount of the fluorophore-labeled collagen adsorbed to the calcium phosphate coating is from 5 μg/cm² to 1 mg/cm². After the fluorophore-labeled collagen has been adsorbed to the pre-substrate, the multi-purpose substrate can be subsequently washed and dried. Exemplary procedures for adsorbing the fluorophore-labeled collagen to the pre-substrate are provided in the Examples section.

The substrates described herein can be used to culture cells. The term “substrate” also encompasses surfaces which permit multiple types of imaging and analysis, including analysis of cultured cells. For example, the substrates described herein can be used for both solution- and image-based analysis of cultured cells. Each of these techniques is described in detail below. The substrates also provide high throughput cell activity assays in real time. Therefore, both solution- and image-based assays can be performed with one substrate, which ultimately reduces material and labor costs.

The substrates described herein are useful in evaluating the activity of a cell or cell precursor. In one aspect, the method comprises (a) culturing cells or cell precursors in a culture medium on the substrate; (b) imaging resorption pits present on the substrate; and/or (c) detecting the presence or absence of a fluorescence signal in a sample of culture medium. In an additional aspect, the method comprises (a) culturing cells or cell precursors in a culture medium on the substrate; (b) imaging the cells or cell precursors present on the substrate; and/or (c) imaging resorption pits present on the substrate; and/or (d) detecting the presence or absence of a fluorescence signal in a sample of culture medium.

The term “activity” is defined herein as any property, function, or mechanism of the cultured cells or cell precursors that can be qualitatively and/or quantitatively measured using the methods described herein. For example, the activity can be the ability of the cells to form resorption pits on the calcium phosphate coating of the substrate. Resorption pits are formed when a cell such as, for example, osteoclasts release hydrogen ions that may dissolve the calcium phosphate coating. Upon dissolution, the cell forms a pit or indentation in the calcium phosphate coating, which can be imaged by SEM or optical microscopy. The ability to effectively quantify the resorption pits (e.g., pit area, number of pits, etc.) is one way to evaluate the ability of cells to adhere and resorb to the substrate. In other aspects, the substrates can be used to evaluate the ability of cancer cells to degrade collagen on the surface of the substrate by monitoring using the fluorophore signal over time.

In addition to imaging cells adhered to the substrate, the substrate can be used for solution-based detection. Not wishing to be bound by theory, when the cell comes into contact with the fluorophore-labeled calcium phosphate substrate, labeled calcium phosphate fragments are produced and released into solution. The fluorophore-labeled calcium phosphate fragments can be detected by methods known in the art for detecting the particular fluorophore used. For example, if the calcium phosphate is labeled with fluorescein, its fluorescence can be detected by use of a fluorimeter with excitation and emission wavelengths of 485 and 535 nm, respectively. Other fluorophores will have their own unique excitation and emission maxima, and these are known in the art. Some types of fluorophores, such as quantum dots, can be imaged by use of image analysis systems that detect fluorescence.

Fluorescence can be detected by time delay methods, which can reduce or eliminate the contribution of non-specific background fluorescence. For example, time-resolved fluorimetry can be used. Devices suitable for carrying out time-resolved fluorimetry include, but are not limited to, a Victor spectrofluorimeter (e.g., Victor or Victor²™ from EG&G Wallac), SPECTRAmax GEMINI (Molecular Devices), the LJL-Analyst, and FLUOstar from BMG Lab Technologies.

Many types of cells can be cultured on the substrate including, but not limited to, stem cells, pluripotent cells, committed stem cells, differentiated cells, bone cells and tumor cells. Examples of stem cells include, but are not limited to, embryonic stem cells, adult stem cells, inducible pluripotent cells, bone marrow stem cells, and umbilical cord stem cells. Other examples of cells used in various embodiments include, but are not limited to, myoblasts, neuroblasts, fibroblasts, glioblasts, germ cells, hepatocytes, chondrocytes, keratinocytes, smooth muscle cells, cardiac muscle cells, connective tissue cells, glial cells, epithelial cells, endothelial cells, hormone-secreting cells, cells of the immune system, and neurons. In one aspect, bone cells such as osteoclasts, osteocytes, and osteoblasts can be cultured with the substrates described herein.

Cells useful herein can be cultured in vitro, derived from a natural source, genetically engineered, or produced by any other means. Any natural source of prokaryotic or eukaryotic cells can be used. Primary cells can be used.

Atypical or abnormal cells such as tumor cells can also be used herein. Tumor cells cultured on the multi-purpose substrates can provide more accurate representations of the native tumor environment in the body for the assessment of drug treatments. Growth of tumor cells on the multi-purpose substrates described herein can facilitate characterization of biochemical pathways and activities of the tumor, including gene expression, receptor expression, and polypeptide production, in an in vivo-like environment allowing for the development of drugs that specifically target the tumor.

Cells that have been genetically engineered can also be used herein. The engineering involves programming the cell to express one or more genes, repressing the expression of one or more genes, or both. Genetic engineering can involve, for example, adding or removing genetic material to or from a cell, altering existing genetic material, or both. Embodiments in which cells are transfected or otherwise engineered to express a gene can use transiently or permanently transfected genes, or both. Gene sequences may be full or partial length, cloned or naturally occurring.

The substrates described herein can comprise one or more bioactive molecules that can facilitate cell adhesion to the calcium phosphate coating, promote cell function, or cell growth, or all three. In one aspect, one or more bioactive molecules are part of the composition used to produce the calcium phosphate coating. In this aspect, the bioactive molecule is dispersed uniformly throughout the calcium phosphate coating. In another aspect, once the fluorescently-labeled calcium phosphate coating has been produced, the coating is contacted with one or bioactive molecules, in addition to collagen.

Bioactive molecules include human or veterinary therapeutics, nutraceuticals, vitamins, salts, electrolytes, amino acids, peptides, polypeptides, proteins, carbohydrates, lipids, polysaccharides, nucleic acids, nucleotides, polynucleotides, glycoproteins, lipoproteins, glycolipids, glycosaminoglycans, proteoglycans, growth factors, differentiation factors, hormones, neurotransmitters, pheromones, chalones, prostaglandins, immunoglobulins, monokines and other cytokines, humectants, minerals, electrically and magnetically reactive materials, light sensitive materials, anti-oxidants, molecules that may be metabolized as a source of cellular energy, antigens, and any molecules that can cause a cellular or physiological response. Any combination of molecules can be used, as well as agonists or antagonists of these molecules. Glycoaminoglycans include glycoproteins, proteoglycans, and hyaluronan. Polysaccharides include cellulose, starch, alginic acid, chytosan, or hyaluronan. Cytokines include, but are not limited to, cardiotrophin, stromal cell derived factor, macrophage derived chemokine (MDC), melanoma growth stimulatory activity (MGSA), macrophage inflammatory proteins 1 alpha (MIP-1 alpha), 2, 3 alpha, 3 beta, 4 and 5, interleukin (IL) 1, IL-2, IL-3, IL-4, IL-5, IL-6, IL-7, IL-8, IL-9, IL-10, IL-11, IL-12, IL-13, TNF-alpha, and TNF-beta. Immunoglobulins useful herein include, but are not limited to, IgG, IgA, IgM, IgD, IgE, and mixtures thereof. Amino acids, peptides, polypeptides, and proteins can include any type of such molecules of any size and complexity as well as combinations of such molecules. Examples include, but are not limited to, structural proteins, enzymes, and peptide hormones.

The term bioactive molecule also includes fibrous proteins, adhesion proteins, adhesive compounds, deadhesive compounds, and targeting compounds. Fibrous proteins include collagen and elastin. Adhesion/deadhesion compounds include fibronectin, laminin, thrombospondin and tenascin C. Adhesive proteins include actin, fibrin, fibrinogen, fibronectin, vitronectin, laminin, cadherins, selectins, intracellular adhesion molecules 1, 2, and 3, and cell-matrix adhesion receptors including but not limited to integrins.

The term bioactive molecule also includes leptin, leukemia inhibitory factor (LIF), RGD peptide, tumor necrosis factor alpha and beta, endostatin, angiostatin, thrombospondin, osteogenic protein-1, bone morphogenic proteins 2 and 7, osteonectin, somatomedin-like peptide, osteocalcin, interferon alpha, interferon alpha A, interferon beta, interferon gamma, interferon 1 alpha, and interleukins 2, 3, 4, 5 6, 7, 8, 9, 10, 11, 12, 13, 15, 16, 17 and 18.

The term “growth factor” as used herein means a bioactive molecule that promotes the proliferation of a cell or tissue. Growth factors useful herein include, but are not limited to, transforming growth factor-alpha. (TGF-alpha), transforming growth factor-beta. (TGF-beta), platelet-derived growth factors including the AA, AB and BB isoforms (PDGF), fibroblast growth factors (FGF), including FGF acidic isoforms 1 and 2, FGF basic form 2, and FGF 4, 8, 9 and 10, nerve growth factors (NGF) including NGF 2.5s, NGF 7.0s and beta NGF and neurotrophins, brain derived neurotrophic factor, cartilage derived factor, bone growth factors (BGF), basic fibroblast growth factor, insulin-like growth factor (IGF), vascular endothelial growth factor (VEGF), EG-VEGF, VEGF-related protein, Bv8, VEGF-E, granulocyte colony stimulating factor (G-CSF), insulin like growth factor (IGF) I and II, hepatocyte growth factor (HGF), glial neurotrophic growth factor (GDNF), stem cell factor (SCF), keratinocyte growth factor (KGF), transforming growth factors (TGF), including TGFs alpha, beta, beta1, beta2, and beta3, skeletal growth factor, bone matrix derived growth factors, and bone derived growth factors and mixtures thereof. Some growth factors can also promote differentiation of a cell or tissue. TGF, for example, can promote growth and/or differentiation of a cell or tissue. Some preferred growth factors include VEGF, NGFs, PDGF-AA, PDGF-BB, PDGF-AB, FGFb, FGFa, HGF, and BGF.

The term “differentiation factor” as used herein means a bioactive molecule that promotes the differentiation of cells or tissues. The term includes, but is not limited to, neurotrophin, colony stimulating factor (CSF), or transforming growth factor. CSF includes granulocyte-CSF, macrophage-CSF, granulocyte-macrophage-CSF, erythropoietin, and IL-3. Some differentiation factors may also promote the growth of a cell or tissue. TGF and IL-3, for example, can promote differentiation and/or growth of cells.

The term “adhesive compound” as used herein means a bioactive molecule that promotes attachment of a cell or tissue to a fiber surface comprising the adhesive compound. Examples of adhesive compounds include, but are not limited to, fibronectin, vitronectin, and laminin.

The term “deadhesive compound” as used herein means a bioactive molecule that promotes the detachment of a cell or tissue from a fiber comprising the deadhesive compound. Examples of deadhesive compounds include, but are not limited to, thrombospondin and tenascin C.

The term “targeting compound” as used herein means a bioactive molecule that functions as a signaling molecule inducing recruitment and/or attachment of cells or tissues to a fiber comprising the targeting compound. Examples of targeting compounds and their cognate receptors include attachment peptides including RGD peptide derived from fibronectin and integrins, growth factors including EGF and EGF receptor, and hormones including insulin and insulin receptor.

In embodiments, fluorescent calcium phosphate surfaces provide a unique real time course assay for image based osteoclast bone resorption. Conventional image based osteoclast bone resorption assays need to terminate different cell cultures at different time points, the results of bone reorption are not really from the same cell culture set. In embodiments, fluorescently labeled calcium phosphate surfaces can help save the substrate, cells and reagents for time course assay, since one fluorescent HA surface can be used for different time points and the images of bone resorption can be captured at different cell culture stages on the same cell culture well coated with fluorescent HA. In embodiments, fluorescence on the surface increases the contrast for the images of bone resorption assay. No additional staining is needed to enhance the image quality for quantitative image analysis.

In additional embodiments, several fluorochromes (such as, for example, calcein, XO, CB, alizarin complexone, doxycycline, rolitetracycline and others) with different fluorescent colors offer options to coat polystyrene tissue culture surface with different fluorescent colors. The variation of fluorescent color of calcium phosphate surface can be combined with other different fluorescent staining assays. The combination of different fluorochromes on the same HA surface provides multi-colored fluorescent HA coatings. The multicolor fluorescence in the same coating offers options to combine the fluorescent HA surface with other fluorescent assays.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention, which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures and other reaction ranges and conditions that can be used to optimize the product purity and yield obtained from the described process. Only reasonable and routine experimentation will be required to optimize such process conditions.

A 5×SBF solution was prepared without sodium bicarbonate (Table 2). Briefly, NaCl, CaCl₂, MgCl₂ and NaHPO₄ in deionizer (DI) water were added sequentially, dissolving the previous chemical before adding the next one. The salt solution was autoclaved using the liquid-cycle on the autoclave (20 min., 15 psi, 121° C., slow exhaust). The solution of 5×SBF was stored at 4° C.

Calcein or XO was mixed with 5×SBF. The pH of the solution was adjusted to 6-7 by adding sodium bicarbonate. TCT treated 96-well plates were then coated with 100 ul per well of carbonated 5×XSBFsolution containing calcein (5-200 μM), XO (10-300□M), or calcein blue (50-100 □M). The plates were then incubated in an inverted position at 35° C. for 18 hours. After the incubation, the salt solution was removed and the plates subsequently washed 5 times with DI water. A fluorescently-labeled calcium phosphate surface was formed.

Another way to make single color fluorescent HA surface was to coat a pre-made hydroxyapatite surface (Osteo-Assay surface, Corning Incorporated, Corning, N.Y.) with calcein (0.075-750M). After diluting the calcein in dilution buffer, 1001 of diluted staining reagent was then added to the calcium phosphate coated well, shielded from light and incubated at room temperature for 30 minutes. After rinsing 3 times with 200 ml of diluted wash buffer, the coated plates were covered with diluted wash medium or left in dry. Optimal staining concentration of calcein was found to 5-7.5 μM for staining the calcium phosphate surface.

FIGS. 3 and 4 illustrate labeled calcium phosphate surfaces labeled with calcein (FIG. 3), and XO (FIG. 4) FIGS. 3 A, B and C are photographs (SEMS of calcein labeled calcium phosphate surfaces labeled with 20 μm, 30 μm and 80 μm calcein mixed with 5× simulated body fluid (SBF) respectively. A 96-well TCT polystyrene plate was coated with the mixture of calcein and 5×SBF in inverted position at 35° C. for 18 hours. FIGS. 4 A, B and C are photographs (SEMS) of XO labeled calcium phosphate surfaces labeled with 10 μm, 50 μm and 100 μm XO mixed with 5× simulated body fluid (SBF) respectively. Dual color fluorescent HA surfaces were prepared by mixing two types of fluorescent dye into the 5×SBF. For example, the 5×SBF was then mixed with two types of fluorescent dye (e.g.: 5 μM calcein and 300 μm XO) and the pH of the solution was adjusted to 6-7 by adding sodium bicarbonate. 100 ul of the 5×SBF solution containing two types of fluorescent dyes were added into each well of a 96-well plate and incubated in an inverted position at 35° C. for 18 hours. The dual color fluorescent coatings emitted different colors when excited at the corresponding wavelength and did not interfere with each other. For example, the dual coating with calcein and XO fluoresces green fluorescent color at 484/535 nm and fluoresces red fluorescent color at 440/570 nm (data not shown).

Collagen was labeled with Eu using a kit from Perkin Elmer (Delphia Eu-Labelling Reagent 1244-301). As described in the user manual from Perkin Elmer, the free amine group of collagen reacted with the isothiocyanato group of Eu³⁺-chelate of N¹-(p-isothiocyanatobenzyl) diethylenetriamine-N¹,N²,N³,N³-tetraacetic acid. It was purified using Superdex200 prep grade (GE Healthcare). The labeled collagen was characterized using a BCA protein assay from Pierce (#23235). The protein was stored at −20° C. The Eu ions were covalently bound to the collagen and only the enhancement solution was able to release Eu from the collagen for measurement.

Eu-labeled collagen was adsorbed on fluorescent calcium phosphate surfaces by immersing the fluorescently labeled calcium phosphate surfaces plates (substrates) prepared as described above, and tissue culture treated polystyrene (TCT) 96 well plates in PBS solution at room temperature for 2 hrs and subsequent washing with PBS three times. The coating concentration was 80 μg/cm².

### Cell Culture

Human osteoclast precursors and medium were purchased from Lonza (Walkersville, Pa.). Human osteoclast precursors were thawed and rinsed with Lonza Osteoclast Precursor Growth Medium with 10% FBS, 2 mM L-glutamine and 100 units/ml penicillin and streptomycin (Pen/Strep). 10,000 cells per well of 96-well plate were seeded with Osteoclast Differentiation Medium containing 33 ng/ml of MCSF and 66 ng/ml of RANKL at 37° C. in a humidified atmosphere of 5% CO₂ for 4-6 days. Cells were cultured on Eu-collagen coated fluorescently-labeled calcium phosphate (F-HA in FIG. 5) or Eu-collagen coated HA surface (Eu-labeled collagen provided on a calcium phosphate surface that does not have a fluorescent label as a comparative example, “HA” in FIG. 5), at 37° C. in a humidified atmosphere of 5% CO₂ for 5-7 days.

### Solution-Based Bone Resorption Assay

5 ul of cell culture supernatant was sampled out of the same cell set cultured on F-HA surface coated with Eu-collagen at indicated time points and each sample of cell culture supernatants was mixed with 200 ml of DELFIA Enhancement Solution (Perkin Elmer) while human osteoclast precursors were cultured in Osteoclast Differentiation Medium (Lonza) as described in the previous section. Victor™ X4 Multilabel Plate Reader (Perkin Elmer) was used to quantify europium signal in the solution for bone resorption assay. The results showed the increase of europium signal from day 3 to day 7 on both Eu-collagen coated fluoresceltly labeled calcium phosphate (F—CP) surface and Eu-collagen coated calcium phosphate (CP) surface (FIG. 5).

### Image-Based Bone Resorption Assay

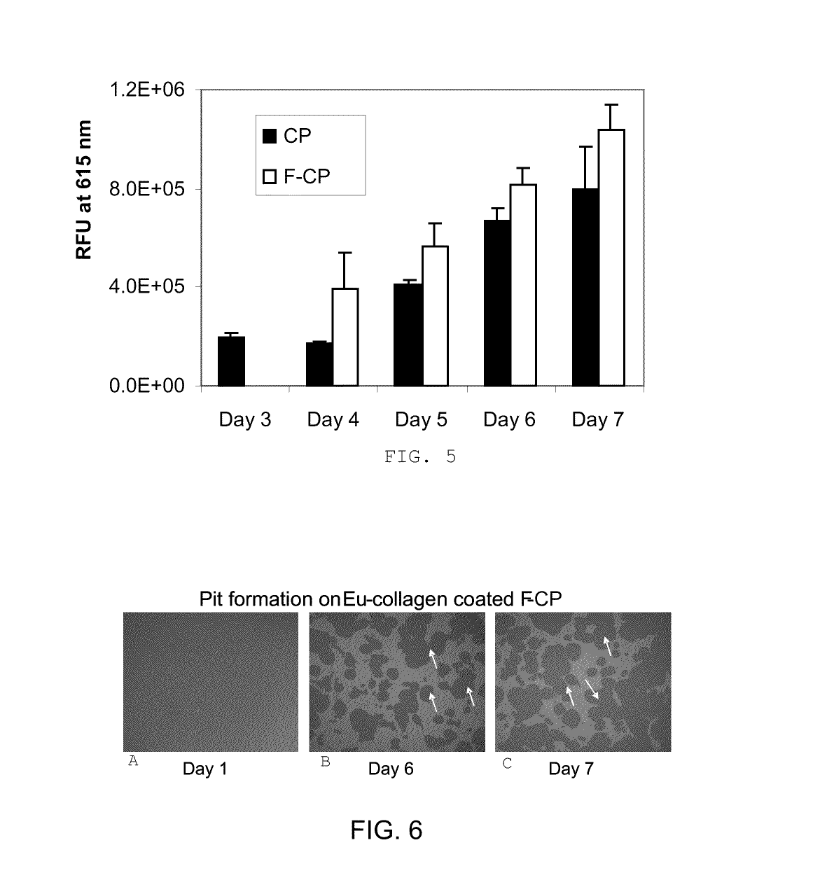

When human osteoclast precursors were cultured on Eu-collagen coated fluorescently labeled calcium phosphate (F-CP) surface in Osteoclast Differentiation medium as described in the above, similar views of the same cell culture well were captured on day 1, day 6 and day 7 by using a Zeiss fluorescent microscope under appropriate excitation/emission wavelengths (492/520 nm) for time real course assay (FIG. 6). The non fluorescent darkened area on the surface (white arrows) indicates the bone resorption pits formation.

Other advantages which are obvious and which are inherent to the invention will be evident to one skilled in the art. It will be understood that certain features and sub-combinations are of utility and may be employed without reference to other features and sub-combinations. This is contemplated by and is within the scope of the claims. Since many possible embodiments may be made of the invention without departing from the scope thereof, it is to be understood that all matter herein set forth or shown in the accompanying drawings is to be interpreted as illustrative and not in a limiting sense.

What is claimed is:

1. A substrate (200) comprising
a. a pre-substrate (100) comprising a calcium phosphate coating (102) on a surface of a base (100) wherein the calcium phosphate coating is labeled with a fluorophore;
b. fluorophore-labeled protein on a surface of the pre-substrate.

2. The substrate of claim 1, wherein the fluorophore labeling the calcium phosphate coating is a calcium chelating fluorophore.

3. The substrate of claim 2, wherein the calcium phosphate coating-fluorophore is selected from the group consisting of calcein, xylenol orange, calcein blue, alizarin complexone, doxycycline, oxytetracycline, rolitetracycline, hematophrophyrin and BAPTA (BAPTA: 1,2-Bis (2-aminophenoxy) ethane-N, N,N′, N′-tetraacetic acid).

4. The substrate of claim 2, wherein the fluorophore is calcein, xylenol orange or calcein blue.

5. The substrate of claim 1, wherein the calcium phosphate coating is labeled with a plurality of fluorophores.

6. The substrate of claim 5 wherein the plurality of fluorophores are selected from the group consisting of calcein, xylenol orange, calcein blue, alizarin complexone, doxycycline, oxytetracycline, rolitetracycline, hematophrophyrin and BAPTA (BAPTA: 1,2-Bis (2-aminophenoxy) ethane-N,N, N′, N′-tetraacetic acid) or a combination.

7. The substrate of claim 5 wherein the plurality of fluorophores comprise calcein and one or more of xylenol orange, calcein blue, alizarin complexone, doxycycline, oxytetracycline, rolitetracycline, hematophrophyrin and BAPTA (BAPTA: 1,2-Bis (2-aminophenoxy) ethane-N,N, N′, N′-tetraacetic acid).

8. The substrate of claim 5 wherein the plurality of fluorophores comprise xylenol orange and one or more of calcein, calcein blue, alizarin complexone, doxycycline, oxytetracycline, rolitetracycline, hematophrophyrin and BAPTA (BAPTA: 1,2-Bis (2-aminophenoxy) ethane-N,N, N′, N′-tetraacetic acid).

9. The substrate of claim 1 wherein the fluorophore labeling the protein is a lanthanide chelate.

10. The substrate of claim 9, wherein the fluorophore labeling the protein is a europium chelate.

11. The substrate of claim 1, wherein the fluorophore labeling the protein is Eu³⁺-N′-(p-isothiocyanatobenzyl) diethylenetriamine-N¹,N²,N³-tetraacetic acid.

12. The substrate of claim 1, wherein the protein is collagen.

13. The substrate of claim 12, wherein the protein is human collagen.

14. The substrate of claim 1, wherein the protein is absorbed to the surface of the pre-substrate.

15. The substrate of claim 1, wherein the calcium phosphate coating comprises hydroxyapatite or substituted hydroxyapatite.

16. The substrate of claim 1, wherein the base comprises polystyrene, polypropylene, polycarbonate, polyester, or any combination thereof.

17. The substrate of claim 1, wherein the base comprises an inorganic material.

18. The substrate of claim 17, wherein the inorganic material comprises glass, quartz, ceramic, silica, a metal oxide, or any combination thereof.

19. The substrate of claim 1, wherein the base comprises a microwell, dish, or flask.

20. The substrate of claim 1, wherein the substrate is prepared by a method comprising:
(a) introducing the base into a solution comprising a plurality of precursor components for producing the calcium phosphate coating and a fluorphore;
(b) inverting the base relative to the solution; and
(c) incubating the inverted base to produce the fluorophore-labeled calcium phosphate coating on the surface of the base
(d) incubating the fluorphore-labeled calcium phosphate coating with a fluorophore-labeled protein.

21. The method of claim 20, further comprising, after step (c), exposing the fluorphore-labeled calcium phosphate coating on the base to gamma irradiation.

22. A method for producing a multiply fluorescently-labeled calcium phosphate protein coating on the surface of a base, the method comprising the steps of:
(a) providing a fluorophore to a calcium phosphate coating on the surface of the base to provide a fluorophore-labeled calcium phosphate coating and then
(b) providing a fluorophore-labeled protein to the fluorophore-labeled calcium phosphate coating to provide a multiply fluorescently-labeled calcium phosphate protein coating.

23. A method for evaluating the activity of a cell or cell precursor, the method comprising
(a) culturing cells or cell precursors in a culture medium on the substrate of claim 1;
(b) exposing the cultured cells on the substrate to a wavelength of light which corresponds to the fluorescence of a fluorophore,
(c) characterizing the fluorescence of the fluorophore.

24. The method of claim 23, wherein cell comprises stem cells, committed stem cells, differentiated cells, tumor cells, myoblasts, neuroblasts, fibroblasts, glioblasts, germ cells, hepatocytes, chondrocytes, keratinocytes, smooth muscle cells, cardiac muscle cells, connective tissue cells, glial cells, epithelial cells, endothelial cells, hormone-secreting cells, cells of the immune system, or neurons.

25. The method of claim 23, wherein the cell is a bone cell, and the bone cell is an osteoclast, an osteocyte, or an osteoblast.

26. The method of claim 23, wherein the cell precursor is a bone cell precursor, and the bone cell precursor is monocyte or a macrophage.

27. The method of claim 23, wherein characterizing comprises quantifying resorption pits produced by a bone cell or bone cell precursor.

28. The method of claim 23, wherein the base comprises a polymer comprising polystyrene, polypropylene, polycarbonate, polyester, or any combination thereof.

29. The method of claim 23, wherein the base comprises a glass slide, wherein a gasket is adhered to the slide to provide at least one temporary well, and adding solution to the well or wells.
